(19) United States
(12) Patent Application Publication
(10) Pub. No.: US 2013/0017539 A1
(43) Pub. Date: Jan. 17, 2013

(54) METHODS AND COMPOSITIONS FOR CHLAMYDIA TRACHOMATIS DIAGNOSTIC TESTING

(71) Applicants: Mitra Choudhury Singhal, Edmonds, WA (US); Cori Anne Barfield, Seattle, WA (US); Kathryn Watts Weaver, Washington, DC (US)

(72) Inventors: Mitra Choudhury Singhal, Edmonds, WA (US); Cori Anne Barfield, Seattle, WA (US); Kathryn Watts Weaver, Washington, DC (US)

(73) Assignee: PROGRAM FOR APPROPRIATE TECHNOLOGY IN HEALTH, Seattle, WA (US)

(21) Appl. No.: 13/309,363

(22) Filed: Dec. 1, 2011

Related U.S. Application Data
(60) Provisional application No. 61/506,393, filed on Jul. 11, 2011.

(51) Int. Cl. C12Q 1/68; C07H 21/04

(52) U.S. Cl. 435/6.11; 536/23.1

(57) ABSTRACT

The invention provides methods, reagent, and kits for detecting the presence of Chlamydia trachomatis in a test sample.

### CROSS-REFERENCE TO RELATED APPLICATION

This application claims the benefit of U.S. Provisional Application No. 61/506,393, filed Jul. 11, 2011.

### STATEMENT OF GOVERNMENT LICENSE RIGHTS

This invention was made with Government support under Grant Number U01 AI070801-05 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 38263_SEQ_Final_{—}2011-12-01.txt. The text file is 14 KB; was created on Dec. 1, 2011; and is being submitted via EFS-Web with the filing of the specification.

### BACKGROUND

Chlamydia trachomatis (“C. trachomatis”) is a bacterial infection that is transmitted mainly by sexual contact. C. trachomatis is the most commonly reported sexually transmitted infection in the United States, with 1,210,523 infections reported to the Center for Disease Control in 2008. Estimates of infection rates are significantly higher than report rates, as most individuals with C. trachomatis infection are unaware of their infection, and thus unlikely to seek treatment. In the developing world, infection rates vary greatly by geographic location and population. Accurate estimates of global prevalence are challenging because few developing countries operate routine screening programs and available figures seldom reflect true levels of infection.

C. trachomatis infection can lead to serious complications. Ascending infection in women can lead to the development of acute pelvic inflammatory disease (PID), frequently in the form of silent endometritis. Symptomatic PID is estimated to occur in 10 to 40 percent of women with untreated C. trachomatis. Additionally, C. trachomatis is thought to be the most common infectious etiologic cause of tubal infertility worldwide, as the organism elicits a chronic inflammatory process that results in tubal occlusion. Other serious adverse outcomes of this process include ectopic pregnancy and chronic pelvic pain. Infection with C. trachomatis also increases indices associated with enhanced likelihood of women transmitting or acquiring HIV/AIDS.

Early diagnosis of C. trachomatis infection is important to reduce the likelihood of transmission and to initiate treatment regimens before clinical complications present. A number of commercially available nucleic acid amplification tests (NAAT) have been developed and are presently the most accurate tests for the diagnosis and screening of C. trachomatis infection. These include the Roche Amplicor® test, the Becton Dickinson ProbeTec™ SDA assay, and the Abbott LCx® assay, all of which target regions of the 7.5 kb cryptic plasmid; the GenProbe Aptima Combo 2® test targeting 16S ribosomal RNA gene (16S rRNA), and the Cepheid GeneExpert® assay.

A major challenge facing the design of NAATs for the detection of C. trachomatis lies in the need to ensure highly sensitive yet specific detection of low burden C. trachomatis infections. The infectious extracellular form of C. trachomatis, the elementary body, is commonly found in low numbers at the site of infection. Therefore, there remains a need for a test for the detection of C. trachomatis that is sensitive and consistently detects low copy infections.

### SUMMARY

In accordance with the foregoing, in one aspect, the invention provides a method for determining the presence of Chlamydia trachomatis (“C. trachomatis”) in a test sample. The method according to this aspect of the invention comprises (a) contacting a test sample with a composition comprising at least one primer pair comprising a forward primer and a reverse primer capable of hybridizing to a target region of the C. trachomatis intergenic spacer sequence separating the 16S and 23S genes consisting of SEQ ID NO:2 to form a reaction mixture; and (b) subjecting said reaction mixture to amplification conditions suitable to amplify at least a portion of said target region.

In another aspect, the invention provides a method for determining the presence of C. trachomatis in a test sample. The method according to this aspect of the invention comprises (a) contacting a test sample with a composition comprising at least one primer pair comprising a forward primer and a reverse primer capable of hybridizing to a target region of the C. trachomatis cryptic plasmid consisting of SEQ ID NO:7 to form a reaction mixture; and (b) subjecting said reaction mixture to amplification conditions suitable to amplify at least a portion of said target region.

In another aspect, the invention provides a method for determining the presence of C. trachomatis in a test sample, said method comprising the steps of (a) contacting a test sample with a composition comprising at least one primer pair comprising a forward primer and a reverse primer capable of hybridizing to a target region of the C. trachomatis intergenic spacer sequence separating the 16S and 23S genes consisting of SEQ ID NO:2 and at least one primer pair comprising a forward primer and a reverse primer capable of hybridizing to a target region of the C. trachomatis cryptic plasmid consisting of SEQ ID NO:7 to form a reaction mixture; and (b) subjecting said reaction mixture to amplification conditions suitable to amplify at least a portion of said target region.

In another aspect, the invention provides a set of oligonucleotides for use in amplifying a target region of nucleic acid derived from the intergenic spacer sequence separating the 16S and 23S genes of C. trachomatis, the set of oligonucleotides comprising a forward primer and a reverse primer, each primer having a target binding region up to 30 nucleotides in length which contains at least 10 contiguous nucleotides that are perfectly complementary to an at least 10 contiguous nucleotide region present in a target sequence consisting of SEQ ID NO:2.

In another aspect, the invention provides a set of oligonucleotides for use in amplifying a target region of nucleic acid derived from the cryptic plasmid of C. trachomatis, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:7.

In another aspect, the invention provides an oligonucleotide for use in amplifying a target region of nucleic acid derived from C. trachomatis, said oligonucleotide having a target binding region of up to 30 bases in length, which stably hybridizes to a target sequence selected from the group consisting of SEQ ID NO:2 and SEQ ID NO:7.

In another aspect, the invention provides a kit for detecting the presence of C. trachomatis in a test sample. In accordance with this aspect of the invention, the kit comprises (a) at least one oligonucleotide comprising a target binding region sequence selected from the group consisting of SEQ ID NO:3, SEQ ID NO:4, SEQ ID NO:8, and SEQ ID NO:9; (b) amplification reagents; and (c) written instructions describing amplification conditions suitable to detect the presence of C. trachomatis in a test sample.

The invention thus provides methods, reagents, and kits for determining the presence of C. trachomatis in a test sample.

### SEQUENCE LISTING

SEQ ID NO:1: C. trachomatis intergenic spacer region full length (Genbank Ref. U68441, incorporated herein by reference)

SEQ ID NO:2: C. trachomatis intergenic spacer target region (nt 91-380 of SEQ ID NO:1)

SEQ ID NOS:3-5: primers and probes for intergenic spacer assay

SEQ ID NO:6: C. trachomatis cryptic plasmid full length (Genbank Ref. X06707, incorporated herein by reference)

SEQ ID NO:7: C. trachomatis cryptic plasmid target region (nt 3675-3743 of SEQ ID NO:6)

SEQ ID NOS:8-10: primers and probes for cryptic plasmid assay

### DETAILED DESCRIPTION

Unless specifically defined herein, all terms used herein have the same meaning as they would to one skilled in the art of the present invention. Practitioners are particularly directed to Sambrook et al. Molecular Cloning: A Laboratory Manual, 2d ed., Cold Spring Harbor Press, Plainsview, N.Y., 1989; and Ausubel et al., Current Protocols in Molecular Biology, John Wiley & Sons, New York, 1999, for definitions and terms of art.

The following definitions are provided in order to provide clarity with respect to the terms as they are used in the specification and claims to describe the present invention.

The term “specifically hybridize” as used herein refers to the ability of a nucleic acid to bind detectably and specifically to a second nucleic acid. Polynucleotides specifically hybridize with target nucleic acid strands under hybridization and wash conditions that minimize appreciable amounts of detectable binding to non-specific nucleic acids. Stringent conditions that can be used to achieve specific hybridization are known in the art.

A “target sequence” or “target nucleic acid sequence” as used herein means a nucleic acid sequence of C. trachomatis, such as a target region of the intergenic spacer sequence (e.g., SEQ ID NO:2), or complement thereof, or a target region of the cryptic plasmid (e.g., SEQ ID NO:7), or complement thereof, that is amplified, detected, or both amplified and detected using one or more of the oligonucleotide primers provided herein. Additionally, while the term target sequence sometimes refers to a double stranded nucleic acid sequence, those skilled in the art will recognize that the target sequence can also be single stranded. In cases where the target is double stranded, polynucleotide primer sequences of the present invention preferably will amplify both strands of the target sequence. As described in Examples 1-2, the primer sequences of the present invention are selected for their ability to specifically hybridize with a range of different C. trachomatis strains.

The term “test sample” as used herein refers to a sample taken from a subject or other source that is suspected of containing or potentially contains a C. trachomatis target sequence. The test sample can be taken from any biological source, such as, for example, tissue, blood, saliva, sputa, mucus, sweat, urine, urethral swabs, cervical swabs, urogenital or anal swabs, conjunctival swabs, ocular lens fluid, cerebral spinal fluid, milk, ascites fluid, synovial fluid, peritoneal fluid, amniotic fluid, fermentation broths, cell cultures, chemical reaction mixtures and the like. The test sample can be used (i) directly as obtained from the source, or (ii) following a pre-treatment to modify the character of the sample. Thus, the test sample can be pre-treated prior to use, for example, by preparing plasma or serum from blood, disrupting cells or viral particles, preparing liquids from solid materials, diluting viscous fluids, filtering liquids, concentrating liquids, inactivating interfering components, adding reagents, purifying nucleic acids, and the like.

The term “label” as used herein means a molecule or moiety having a property or characteristic that is capable of detection and, optionally, of quantitation. A label can be directly detectable, as with, for example (and without limitation), radioisotopes, fluorophores, chemiluminophores, enzymes, colloidal particles, fluorescent microparticles and the like; or a label may be indirectly detectable as with, for example, specific binding members. It will be understood that directly detectable labels may require additional components such as, for example, substrates, triggering reagents, quenching moieties, light, and the like to enable detection and/or quantitation of the label. When indirectly detectable labels are used, they are typically used in combination with a “conjugate.” A conjugate is typically a specific binding member that has been attached or coupled to a directly detectable label. Coupling chemistries for synthesizing a conjugate are well known in the art and can include, for example, any chemical means and/or physical means that do not destroy the specific binding property of the specific binding member or the detectable property of the label. As used herein, “specific binding member” means a member of a binding pair, i.e., two different molecules where one of the molecules, through for example chemical or physical means, specifically binds to the other molecule. In addition to antigen and antibody specific binding pairs, other specific binding pairs include, but are not intended to be limited to, avidin and biotin; haptens and antibodies specific for haptens; complementary nucleotide sequences; enzyme cofactors or substrates and enzymes; and the like.

A polynucleotide, in the context of the present invention, is a nucleic acid polymer of ribonucleic acid (RNA), deoxyribonucleic acid (DNA), modified RNA or DNA, or RNA or DNA mimetics (such as, without limitation, PNAs) and derivatives thereof, and homologues thereof. Thus, polynucleotides include polymers composed of naturally occurring nucleobases, sugars, and covalent internucleoside (backbone) linkages as well as polymers having non-naturally-occurring portions that function similarly. Such modified or substituted nucleic acid polymers are well known in the art, and, for the purposes of the present invention, are referred to as “analogues.” For ease of preparation and familiarity to the skilled artisan, polynucleotides are preferably modified or unmodified polymers of deoxyribonucleic acid or ribonucleic acid.

As used herein, the term “primer” means a polynucleotide that can serve to initiate a nucleic acid chain extension reaction. Typically, primers have a length of 10 to about 50 nucleotides, although primers can be longer than 50 nucleotides.

As used herein, the term “sequence identity” or “percent identical” as applied to nucleic acid molecules is the percentage of nucleic acid residues in a candidate nucleic acid molecule sequence that are identical with a subject nucleic acid molecule sequence (such as the nucleic acid molecule sequence set forth in SEQ ID NO:2), after aligning the sequences to achieve the maximum percent identity, and not considering any nucleic acid residue substitutions as part of the sequence identity. No gaps are introduced into the candidate nucleic acid sequence in order to achieve the best alignment. Nucleic acid sequence identity can be determined in the following manner. The subject polynucleotide molecule sequence is used to search a nucleic acid sequence database, such as the Genbank database, using the program BLASTN version 2.1 (based on Altschul et al., Nucleic Acids Research 25:3389-3402, 1997). The program is used in the ungapped mode. Default filtering is used to remove sequence homologies due to regions of low complexity as defined in J. C. Wootton and S. Federhen, Methods in Enzymology 266:554-571, 1996. The default parameters of BLASTN are utilized.

The present invention further encompasses homologues of the polynucleotides (i.e., primers and detection probes) having nucleic acid sequences set forth in SEQ ID NOS:3-5 and 8-10. As used herein, the term “homologues” refers to nucleic acids having one or more alterations in the primary sequence, set forth in any one of SEQ ID NOS:3-5 and 8-10, that does not destroy the ability of the polynucleotide to specifically hybridize with a target sequence, as described above. Accordingly, a primary sequence can be altered, for example, by the insertion, addition, deletion or substitution of one or more of the nucleotides of, for example, SEQ ID NOS:3-5 and 8-10. Thus, in one embodiment, homologues have a length in the range of from 10 to 30 nucleotides and have a consecutive sequence of at least 10, 11, 12, 13, 14, 15, 16, 17, 18, 19 20, 21, 22, 23, or more nucleotides of the nucleic acid sequences of SEQ ID NOS:3-5 and 8-10 and will retain the ability to specifically hybridize with a target sequence, as described above. Ordinarily, the homologues will have a nucleic acid sequence having at least 85%, 90%, or 95% nucleic acid sequence identity with a nucleic acid sequence set forth in SEQ ID NOS:3-5 and 8-10. In some embodiments, homologues have a length in the range of from 10 to 30 nucleotides and have a nucleotide sequence substantially identical to a nucleic acid sequence set forth as SEQ ID NOS:3-5 and 8-10, with the difference being the presence of 1, 2, or 3 mismatches, provided that the homologues do not contain two or more consecutive mismatches.

The polynucleotides of the present invention thus comprise primers and probes that specifically hybridize to a target sequence of the invention—for example, the nucleic acid molecules having any one of the nucleic acid sequences set forth in SEQ ID NOS:3-5 and 8-10, including analogues and/or derivatives of said nucleic acid sequences and homologues thereof, that can specifically hybridize with a target sequence of the invention. As described below, polynucleotides of the invention can be used as primers and/or probes to amplify or detect C. trachomatis.

The polynucleotides according to the present invention can be prepared by conventional techniques well known to those skilled in the art. For example, the polynucleotides can be prepared using conventional solid-phase synthesis using commercially available equipment, such as that available from Applied Biosystems USA Inc. (Foster City, Calif.), DuPont, (Wilmington, Del.), or Milligen (Bedford, Mass.). Modified polynucleotides, such as phosphorothioates and alkylated derivatives, can also be readily prepared by similar methods known in the art. See, for example, U.S. Pat. Nos. 5,464,746; 5,424,414; and 4,948,882.

The polynucleotides according to the present invention can be employed directly as probes for the detection or quantitation, or both, of C. trachomatis nucleic acids in a test sample.

In one aspect, the methods comprise detecting the presence of a target region of the intergenic spacer region of C. trachomatis in a test sample. The intergenic spacer is a 232 base pair region separating the 16S and 23S ribosomal ribonucleic acid genes. (Everett, K. and Andersen, A., “The Ribosomal Intergenic Spacer and Domain I of the 23S rRNA Gene are Phylogenetic Markers for Chlamydia ssp.,” Int. J of Systemic Biology 47(2):461-73, 1997.) The intergenic spacer region sequence includes partial sequence of 16S ribosomal RNA gene, intergenic spacer, and partial sequence of 23S ribosomal RNA gene. The full-length nucleotide sequence of the intergenic spacer region of C. trachomatis from the reference intergenic spacer region sequence of C. trachomatis (Genbank Ref. U68441) is set forth as SEQ ID NO:1. In one embodiment, the target region consists of SEQ ID NO:2 (nucleotides 91-380 of SEQ ID NO:1).

In accordance with one embodiment of the invention, the method comprises contacting a test sample with a composition comprising at least one primer pair comprising a forward primer and a reverse primer capable of hybridizing to a target region of the intergenic spacer sequence of C. trachomatis consisting of SEQ ID NO:2 to form a reaction mixture and subjecting said reaction mixture to amplification conditions suitable to amplify the target region. The amplification conditions are suitable to allow hybridization between the target sequence and the primer pair. In one embodiment, the composition comprises a primer having a target-binding region consisting of SEQ ID NO:3. In one embodiment, the composition comprises a primer having a target-binding region consisting of SEQ ID NO:4. In some embodiments, the amplified target region is then detected by a probe that hybridizes to the amplified target region using methods well known in the art. In one embodiment, the probe comprises a target-binding region consisting of SEQ ID NO:5.

In one aspect, the methods comprise detecting the presence of a target region of the cryptic plasmid of C. trachomatis in a test sample. The cryptic plasmid of C. trachomatis is a 7.5 kb plasmid. There are approximately 7-10 copies of the plasmid present per C. trachomatis elemental body, making it a highly sensitive target for the detection of C. trachomatis infections. However, some variants of the cryptic plasmid have a 377 base pair deletion, and therefore may not be detected by certain assays. The target region detected by the claimed methods is outside the deleted region of the variant, in order to increase the likelihood of detecting the wild type cryptic plasmid, as well as the variant. The full-length nucleotide sequence of the wild-type cryptic plasmid of C. trachomatis from the reference cryptic plasmid of C. trachomatis sequence (Genbank Ref. X06707) is set forth as SEQ ID NO:6. In one embodiment, the target region consists of SEQ ID NO:7 (nucleotides 3677-3745 of SEQ ID NO:6).

In accordance with one embodiment of the invention, the method comprises contacting a test sample with a composition comprising at least one primer pair comprising a forward primer and a reverse primer capable of hybridizing to a target region of the cryptic plasmid of C. trachomatis consisting of SEQ ID NO:7 to form a reaction mixture and subjecting said reaction mixture to amplification conditions suitable to amplify the target region. The amplification conditions are suitable to allow hybridization between the target sequence and the primer pair. In one embodiment, the composition comprises a primer having a target-binding region consisting of SEQ ID NO:8. In one embodiment, the composition comprises a primer having a target-binding region consisting of SEQ ID NO:9. In some embodiments, the amplified target region is then detected by a probe that hybridizes to the amplified target region using methods well known in the art. In one embodiment, the probe comprises a target-binding region consisting of SEQ ID NO:10.

The polynucleotides (i.e., primers and probes) of the present invention may incorporate one or more detectable labels. Detectable labels are molecules or moieties having a property or characteristic that can be detected directly or indirectly and are chosen such that the ability of the polynucleotide to hybridize with its target sequence is not adversely affected. Methods of labeling nucleic acid sequences are well known in the art (see, for example, Ausubel et al., Current Protocols in Molecular Biology, Wiley & Sons, New York, 1997, and updates).

Amplification procedures are well-known in the art and include, but are not limited to, polymerase chain reaction (PCR), TMA, rolling circle amplification, nucleic acid sequence based amplification (NASBA), and strand displacement amplification (SDA). One skilled in the art will understand that for use in certain amplification techniques the primers may need to be modified, for example, for SDA the primer comprises additional nucleotides near its 5′ end that constitute a recognition site for a restriction endonuclease. Similarly, for NASBA the primer comprises additional nucleotides near the 5′ end that constitute an RNA polymerase promoter. Polynucleotides thus modified are considered to be within the scope of the present invention.

Certain criteria are taken into consideration when selecting the primers and probes for use in the methods of the invention. For example, for primer pairs for use in the amplification reactions, the primers are selected such that the likelihood of forming 3′ duplexes is minimized, and such that the melting temperatures (Tm) are sufficiently similar to optimize annealing to the target sequence and minimize the amount of non-specific annealing. In this context, the polynucleotides according to the present invention are provided in combinations that can be used as primers in amplification reactions to specifically amplify target nucleic acid sequences.

The amplification method of the present invention generally comprises (a) forming a reaction mixture comprising nucleic acid amplification reagents, at least one set of primers of the present invention, and a test sample suspected of containing an at least one target sequence, and (b) subjecting the mixture to amplification conditions to generate at least one copy of a nucleic acid sequence complementary to the target sequence.

Step (b) of the above methods can be repeated any suitable number of times (prior to detection of the amplified region), e.g., by thermal cycling the reaction mixture between 10 and 100 times, typically between about 20 and about 60 times, more typically between about 25 and about 45 times, such as between about 30 and 40 times.

Nucleic acid amplification reagents include reagents that are well known and may include, but are not limited to, an enzyme having at least polymerase activity, enzyme cofactors such as magnesium or manganese; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

Amplification conditions are conditions that generally promote annealing and extension of one or more nucleic acid sequences. It is well known that such annealing is dependent in a rather predictable manner on several parameters, including temperature, ionic strength, sequence length, complementarity, and G:C content of the sequences. For example, lowering the temperature in the environment of complementary nucleic acid sequences promotes annealing. For any given set of sequences, melt temperature (or Tm) can be estimated by any of several known methods. Typically, diagnostic applications utilize hybridization temperatures that are about 10° C. (e.g., 2° C. to 18° C.) below the melt temperature. Ionic strength or “salt” concentration also impacts the melt temperature, since small cations tend to stabilize the formation of duplexes by negating the negative charge on the phosphodiester backbone. Typical salt concentrations depend on the nature and valency of the cation, but are readily understood by those skilled in the art. Similarly, high G:C content and increased sequence length are also known to stabilize duplex formation, because G:C pairings involve 3 hydrogen bonds where A:T pairs have just two, and because longer sequences have more hydrogen bonds holding the sequences together. Thus, a high G:C content and longer sequence lengths impact the hybridization conditions by elevating the melt temperature.

Specific amplicons produced by amplification of target nucleic acid sequences using the polynucleotides of the present invention, as described above, can be detected by a variety of methods known in the art. For example, one or more of the primers used in the amplification reactions may be labeled such that an amplicon can be directly detected by conventional techniques subsequent to the amplification reaction. Alternatively, a probe consisting of a labeled version of one of the primers used in the amplification reaction, or a third polynucleotide distinct from the primer sequences that has been labeled and is complementary to a region of the amplified sequence can be added after the amplification reaction is complete. The mixture is then submitted to appropriate hybridization and wash conditions and the label is detected by conventional methods.

The amplification product produced as above can be detected during or subsequent to the amplification of the target sequence. Methods for detecting the amplification of a target sequence during amplification are outlined above, and described, for example, in U.S. Pat. No. 5,210,015. Gel electrophoresis can be employed to detect the products of an amplification reaction after its completion. Alternatively, amplification products are hybridized to probes, then separated from other reaction components, and detected using microparticles and labeled probes.

It will be readily appreciated that a procedure that allows both amplification and detection of target nucleic acid sequences to take place concurrently in a single unopened reaction vessel would be advantageous. Such a procedure would avoid the risk of “carry-over” contamination in the post-amplification processing steps, and would also facilitate high-throughput screening or assays and the adaptation of the procedure to automation. Furthermore, this type of procedure allows “real-time” monitoring of the amplification reaction as well as more conventional “end-point” monitoring.

The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively, a third polynucleotide distinct from the primer sequences, which is complementary to a region of the amplified sequence, may be included in the reaction, as when a primer/probe set of the invention is used.

For use in an assay as described above, in which both amplification with polynucleotide primers and detection of target sequences using a polynucleotide probe occur concurrently in a single unopened reaction vessel, the polynucleotide probe preferably possesses certain properties. For example, since the probe will be present during the amplification reaction, it should not interfere with the progress of this reaction, and should also be stable under the reaction conditions. In addition, for real-time monitoring of reactions, the probe should be capable of binding its target sequence under the conditions of the amplification reaction, and to emit a signal only upon binding this target sequence. Examples of probe molecules that are particularly well suited to this type of procedure include molecular beacon probes and probes comprising a fluorophore covalently attached to the 5′ end of the probe and a quencher at the 3′ end (e.g., TaqMan® probes).

The present invention, therefore, contemplates the use of the polynucleotides as TaqMan® probes. As is known in the art, TaqMan® probes are dual-labeled fluorogenic nucleic acid probes composed of a polynucleotide complementary to the target sequence that is labeled at the 5′ terminus with a fluorophore and at the 3′ terminus with a quencher. TaqMan® probes are typically used as real-time probes in amplification reactions. In the free probe, the close proximity of the fluorophore and the quencher ensures that the fluorophore is internally quenched. During the extension phase of the amplification reaction, the probe is bound to the target sequence, cleaved by the 5′ nuclease activity of the polymerase and the fluorophore is released. The released fluorophore can then fluoresce and thus produces a detectable signal.

Suitable fluorophores and quenchers for use with the polynucleotides of the present invention can be readily determined by one skilled in the art (see also, Tyagi et al., Nature Biotechnol. 16:49-53, 1998; Marras et al., Genet. Anal.: Biomolec. Eng. 14:151-156, 1999). Many fluorophores and quenchers are available commercially, for example, from Molecular Probes (Eugene, Oreg.) or Biosearch Technologies, Inc. (Novato, Calif.). Examples of fluorophores that can be used in the present invention include, but are not limited to, fluorescein and fluorescein derivatives such as carboxy fluorescein (FAM®), a dihalo-(C1 to C8)dialkoxycarboxyfluorescein, 5-(2′-aminoethyl)aminonaphthalene-1-sulphonic acid (EDANS), coumarin and coumarin derivatives, Lucifer yellow, Texas red, tetramethylrhodamine, tetrachloro-6-carboxyfluoroscein, 5-carboxyrhodamine, cyanine dyes and the like. Quenchers include, but are not limited to, DABCYL, 4′-(4-dimethylaminophenylazo)benzoic acid (DAB SYL), 4-dimethylaminophenylazophenyl-4-dimethylaminophenylazophenyl-4′-maleimide (DABMI), tetramethylrhodamine, carboxytetramethylrhodamine (TAMRA), dihydrocyclopyrroloindole tripeptide minor groove binder (MGB®) dyes and the like. Methods of coupling fluorophores and quenchers to nucleic acids are well known in the art. The present invention thus includes the use of the polynucleotides in a method to specifically amplify and detect target nucleic acid sequences in a test sample in a single tube format. This may be achieved, for example, by including in the reaction vessel an intercalating dye such as SYBR Green or an antibody that specifically detects the amplified nucleic acid sequence. Alternatively, a third polynucleotide distinct from the primer sequences, which is complementary to a region of the amplified sequence, may be included in the reaction, as when a primer/probe set of the invention is used.

In accordance with the present invention, therefore, the combinations of two primers and at least one probe, as described above, can be used in either end-point amplification and detection assays, in which the strength of the detectable signal is measured at the conclusion of the amplification reaction, or in real-time amplification and detection assays, in which the strength of the detectable signal is monitored throughout the course of the amplification reaction.

The polynucleotides according to the present invention can also be used in assays to detect the presence and/or quantitate the amount of C. trachomatis nucleic acid present in a test sample. Thus, the polynucleotides according to the present invention can be used in a method to specifically amplify, detect, and quantitate target nucleic acid sequences in a test sample, which generally comprises the steps of (a) forming a reaction mixture comprising nucleic acid amplification reagents, at least one polynucleotide probe sequence that incorporates a label that produces a detectable signal upon hybridization of the probe to its target sequence, at least one polynucleotide primer, and a test sample that contains one or more target nucleic acid sequences; (b) subjecting the mixture to amplification conditions to generate at least one copy of the target nucleic acid sequence, or a nucleic acid sequence complementary to the target sequence; (c) hybridizing the probe to the target nucleic acid sequence or the nucleic acid sequence complementary to the target sequence, so as to form a probe:target hybrid; (d) detecting the probe:target hybrid by detecting the signal produced by the hybridized labeled probe; and (e) comparing the amount of the signal produced to a standard as an indication of the amount of target nucleic acid sequence present in the test sample.

One skilled in the art will understand that, as outlined above, step (b) of the above method can be repeated several times prior to step (c) by thermal cycling the reaction mixture by standard techniques known in the art.

Various types of standards for quantitative assays are known in the art. For example, the standard can consist of a standard curve compiled by amplification and detection of known quantities of C. trachomatis nucleic acids under the assay conditions. Alternatively, an internal standard can be included in the reaction. Such internal standards generally comprise a control target nucleic acid sequence and a control polynucleotide probe. The internal standard can optionally further include an additional pair of primers. The primary sequence of these control primers may be unrelated to the polynucleotides of the present invention and specific for the control target nucleic acid sequence.

In another aspect, the invention provides a set of oligonucleotides for use in amplifying a target region of nucleic acid derived from the intergenic spacer sequence of C. trachomatis, the set of oligonucleotides comprising a forward primer and a reverse primer, each primer having a target binding region. In some embodiments, the target binding region is located at the 3′ end of the oligonucleotide. In some embodiments, the target binding region is from 10 to 30 nucleotides in length and contains at least 10 contiguous nucleotides that are perfectly complementary to an at least 10 contiguous nucleotide region present in a target sequence consisting of SEQ ID NO:2. In one embodiment, the forward primer comprises a target-binding region consisting of SEQ ID NO:3. In one embodiment, the reverse primer comprises a target-binding region consisting of SEQ ID NO:4. In one embodiment, the detection probe comprises a target-binding region consisting of SEQ ID NO:5.

In another aspect, the invention provides a set of oligonucleotides for use in amplifying a target region of nucleic acid derived from the cryptic plasmid of C. trachomatis, the set of oligonucleotides comprising a forward primer and a reverse primer, each primer having a target binding region. In some embodiments, the target binding region is located at the 3′ end of the oligonucleotide. In some embodiments, the target binding region is from 10 to 30 nucleotides in length, and contains at least 10 contiguous nucleotides that are perfectly complementary to an at least 10 contiguous nucleotide region present in a target sequence consisting of SEQ ID NO:7. In one embodiment, the forward primer comprises a target-binding region consisting of SEQ ID NO:8. In one embodiment, the reverse primer comprises a target-binding region consisting of SEQ ID NO:9. In one embodiment, the detection probe comprises a target-binding region consisting of SEQ ID NO:10.

In another aspect, the invention provides an oligonucleotide for use in amplifying a target region of nucleic acid derived from C. trachomatis, said oligonucleotide having a target binding region. In some embodiments, the target binding region is located at the 3′ end of the oligonucleotide. In some embodiments, the target binding region is from 10 to 30 bases in length, and stably hybridizes to a target sequence selected from the group consisting of SEQ ID NO:2 and SEQ ID NO:7. In one embodiment, the oligonucleotide comprises a target-binding region that contains at least 10 contiguous nucleotides that are perfectly complementary to at least 10 contiguous nucleotides in said target sequence.

In another aspect, the invention provides a kit for determining the presence of C. trachomatis in a test sample. In accordance with this aspect of the invention, the kit comprises (a) at least one oligonucleotide comprising a target binding region sequence selected from the group consisting of SEQ ID NO:3, SEQ ID NO:4, SEQ ID NO:5, SEQ ID NO:8, SEQ ID NO:9, and SEQ ID NO:10; (b) amplification reagents; and (c) written instructions describing amplification conditions suitable to detect the presence of C. trachomatis in a test sample.

In one embodiment, kits for the detection of C. trachomatis nucleic acids may additionally contain a control target nucleic acid and a control polynucleotide probe. Thus, in one embodiment of the present invention, the kits comprise one of the above combinations of polynucleotides comprising at least two primers and at least one probe, together with a control target nucleic acid sequence, which can be amplified by the specified primer pair, and a control polynucleotide probe. The present invention further provides kits that include control primers, which specifically amplify the control target nucleic acid sequence.

The kits can optionally include amplification reagents, reaction components, and/or reaction vessels. Typically, at least one sequence bears a label, but detection is possible without this. Thus, one or more of the polynucleotides provided in the kit may have a detectable label incorporated, or the kit may include reagents for labeling the polynucleotides. One or more of the components of the kit may be lyophilized and the kit may further comprise reagents suitable for the reconstitution of the lyophilized components.

The polynucleotides, methods, and kits of the present invention are useful in clinical or research settings for the detection and/or quantitation of C. trachomatis nucleic acids. Thus, in these settings, the polynucleotides can be used in assays to diagnose C. trachomatis infection in a subject, or to monitor the quantity of a C. trachomatis target nucleic acid sequence in a subject infected with C. trachomatis.

The following examples merely illustrate the best mode now contemplated for practicing the invention, but should not be construed to limit the invention.

### EXAMPLES

### Example 1

This Example describes the rationale for selection of the target region of the intergenic spacer sequence of C. trachomatis to be used in a diagnostic test.

Methods:

The target region was selected following construction of multiple sequence alignments, including sequences of different strains of C. trachomatis and also sequences from near neighbor organisms, C. pneumoniae and C. psittaci.

Alignments incorporated the intergenic spacer sequence of C. trachomatis strains with the following Genbank/DMBL accession numbers: D89067.1, D85722.1, D88316.1, D85721.1, D85720.1, D85719.1, DQ019298.1, DQ019291.1, DQ019297.1, DQ019296.1, DQ019300.1, DQ019308.1, DQ019309.1, DQ019301.1, DQ019310.1, DQ019299.1, DQ019307.1, DQ019306.1, DQ019305.1, DQ019304.1, DQ019293.1, DQ019292.1, DQ019303.1, DQ019302.1, DQ019295.1, DQ019294.1, U68440.1, U68442.1, U68441.1, and U68438.1.

Alignments incorporated the intergenic spacer sequence of C. pneumoniae strains with the following Genbank/DMBL accession numbers: L06108.1, DQ444323.1, U68426, U68422.1, U68423.1, U68421.1, 068424.1, U68425 and U76711.2

Alignments incorporated the intergenic spacer sequence of C. psittaci strains with the following Genbank/DMBL accession numbers: U68447.2, D85711.1, D85713.1, D85712.1, U68419.2, AF481048.1, AF481050.1, AF481052.1, AF481049.1, U68450.1, U68449.1, U68448.1, U68453.1, U68456.1, AF481051.1, U68454.1, U68452.1, U68455.1 and EF165622

After selection of the target region, primers and probes were designed in a Taqman-MGB format. A BLAST search was conducted to determine if there was cross-reactivity of the sequences to other organisms likely to be present at the site of sample collection. Selected sequences were then tested using C. trachomatis serovar H as a reference strain. Due to the high degree of sequence homology in the intergenic spacer sequences of C. trachomatis, C. pneumoniae, and C. psittaci, a second assay is necessary to specifically identify C. trachomatis in a test sample.

### Example 2

This Example describes the rationale for selection of the target region of the cryptic plasmid of C. trachomatis to be used in a diagnostic test.

Methods:

The target region was selected following construction of multiple sequence alignments, including sequences from all eight open reading frames of C. trachomatis cryptic plasmid, and also cryptic plasmid sequences from near neighbor organisms, C. pneumoniae and C. psittaci.

C. trachomatis cryptic plasmid Open Reading Frame 2 was selected as the target region. After selection of the target region, primers and probes were designed in a Taqman-MGB format. A BLAST search was conducted to determine if there was cross-reactivity of the sequences to other organisms likely to be present at the site of sample collection.

### Example 3

This Example describes the sequences of the primers and probes and reaction conditions used in a real-time PCR assay targeting the C. trachomatis intergenic spacer sequence.

Methods:

Primers:
Forward Primer
(SEQ ID NO: 3)
5′-GAGTTGGTTTTACCTTAAGTCGTTGACTCAA-3′
Reverse Primer
(SEQ ID NO: 4)
5′-ACCATGATTATTTCTTTACAATAACATACTTGATCA-3′
Probe:
(SEQ ID NO: 5)
5′-6FAM-CAAGGTGAGGCTGATGACTAGGATGA-MGB-3′

The primers and probe were custom ordered from and synthesized by Applied Biosystems (ABI) of Carlsbad, Calif. A standard reaction master mix was also obtained from ABI.

The components of the reaction mixture used in the PCR assay are set forth in Table 1.

TABLE 1

| | | Final |
|---|---|---|
| Component | /single reaction | conc./reaction |
| ABI 2X mastermix* | 12.5 μl | |
| Forward primer (20 μM) | 1 μl | 800 nM |
| Reverse primer (20 μM) | 1 μl | 800 nM |
| Probe (100 μM) | 0.038 μl | 300 nM |
| Template (10 ng gDNA) | 2.5 μl | |
| PCR grade H₂O | 7.962 μl | |
| Total volume | 25 μl | |

*(contains dNTPS, Taq DNA polymerase, MgCl₂ and reaction buffer)

The PCR reaction was performed using an ABI 7300 Realtime PCR system under the following conditions:

The Detector on the instrument was set to “FAM (no quench)”
Initial 50° C., 2-minute incubation for UNG nuclease digestion (optional)
Initial denaturation at 95° C., 10 minutes

| | |
|---|---|
| 40 cycles of | |
| Denaturation | 95° C., 15 sec |
| Anneal/Extension | 60° C., 1 min |

Data was collected in the 60° C. anneal/extension step.

Results, measured in mean cycle-threshold (Ct) values, are provided in Example 5.

### Example 4

This Example describes the sequences of the primers and probes and reaction conditions used in a real-time PCR assay targeting the C. trachomatis cryptic plasmid.

Methods:

Primers:
Forward Primer
(SEQ ID NO: 8)
5′-TTGGCTCAAAATGGGATGGT-3′
Reverse Primer
(SEQ ID NO: 9)
5′-TGCTAATGCATGGTAATGAGATGA-3′
Probe:
(SEQ ID NO: 10)
5′-FAM-AGTTATAGGTCTTGATTTTC-MGB-3′

The primers and probe were custom ordered from and synthesized by Applied Biosystems (ABI) of Carlsbad, Calif. A standard reaction master mix was also obtained from ABI.

The components of the reaction mixture used in the PCR assay are set forth in Table 2.

TABLE 2

| | | Final |
|---|---|---|
| Component | /single reaction | conc./reaction |
| ABI 2X mastermix* | 12.5 μl | |
| Forward primer (20 μM) | 1 μl | 800 nM |
| Reverse primer (20 μM) | 1 μl | 800 nM |
| Probe (100 μM) | 0.038 μl | 300 nM |
| Template (10 ng gDNA) | 2.5 μl | |
| PCR grade H₂O | 7.962 μl | |
| Total volume | 25 μl | |

*(contains dNTPS, Taq DNA polymerase, MgCl₂ and reaction buffer)

The PCR reaction was performed using an ABI 7300 Realtime PCR system under the following conditions:

The Detector on the instrument was set to “FAM (no quench)”
Initial 50° C., 2-minute incubation for UNG nuclease digestion (optional)
Initial denaturation at 95° C., 10 minutes

| | |
|---|---|
| 40 cycles of | |
| Denaturation | 95° C., 15 sec |
| Anneal/Extension | 60° C., 1 min |

Data was collected in the 60° C. anneal/extension step.

Results, measured in mean cycle-threshold (Ct) values, are provided in Example 5.

### Example 5

This Example describes assays conducted to validate the use of the primers, probes, and reaction conditions described in Examples 3 and 4 for the detection of C. trachomatis.

Background/Rationale:

C. trachomatis has a number of sub-types originally classified based on the immune-epitope analysis of the major outer membrane protein (MOMP) using monoclonal and polyclonal antibodies. The 18 groups, or serovars, can be grouped by pathogenicity as follows: serovars A, B, Ba, and C are associated with trachoma, D and K with urogenital infections, and L1, L2, and L3 with lymphogranuloma venereum. They can also be classed by amino acid similarity; classed in this way there are three sub-groups: group B (serovars B, Ba, D, Da, E, L1, L2, and L2a), group C (serovars A, C, H, I, Ia, J, Ja, K, and L3) and an intermediate group (serovars F and G). (Molano et al., “Combination of PCR targeting the VD2 of omp1 and the reverse line blot analysis for typing the urogenital chlamydia trachomatis serovars in cervical scrape specimens,” J. Clin. Microbiol. 42(7):2935-2935, July 2004.) The goal of a successful diagnostic for detection of C. trachomatis is for sensitive and inclusive detection of chlamydial serovars.

Methods:

In this Example, a number of blinded panels of organisms representative of the three major groups of C. trachomatis, B complex, intermediate F/G, and C complex were tested in both the intergenic spacer assay, carried out as described in Example 3, and the cryptic plasmid assay, carried out as described in Example 4. Elemental bodies (EB), the infectious portion of the organism, were harvested and enumerated by counting and diluted to a concentration of 1×10⁶ EBs/ml, and serially diluted. The dilutions were blinded and extracted and also tested in both the intergenic spacer assay, carried out as described in Example 3, and the cryptic plasmid assay, carried out as described in Example 4.

The intergenic spacer assay and cryptic plasmid assay yielded concordant results as shown in Table 3. Quantification of EBs was performed by microscopy and only includes viable EBs in estimates. In contrast, the real-time PCR assays detect DNA from both viable and non-viable C. trachomatis EBs. Therefore, sensitivity values (number of EBs detected per reaction) in Table 3 are approximate.

TABLE 3
Serovars tested in cryptic plasmid and intergenic spacer assays

| | Detected in | | Detected in | |
|---|---|---|---|---|
| C. trachomatis | Cryptic | Sensitivity | Intergenic | Sensitivity |
| serovar | Plasmid assay | (EB/rxn) | Spacer assay | (EB/rxn) |
| D | + | 107 | + | 1075 |
| E | + | 95 | + | 95 |
| F | + | 8 | + | 80 |
| G | + | 1.5 | + | 11.5 |
| H | + | 0.5 | + | 50 |
| I | + | <20 | + | <20 |
| J | + | 1.25 | + | 12.5 |
| K | + | <20 | + | <20 |
| LGV1 | + | <20 | + | <20 |
| LGV2 | + | <20 | + | <20 |
| LGV3 | + | <20 | + | <20 |

The data shown in Table 3 illustrate that the claimed intergenic spacer assay and cryptic plasmid assay can detect the presence of different serovars of C. trachomatis at low levels of EBs.

### Example 6

In this Example, real-time polymerase chain reaction assays were performed on archived, stabilized clinical samples and compared to the results obtained using the commercially available GenProbe Aptima Combo 2® Assay for the detection of C. trachomatis (the GenProbe assay results are measured in relative light units (RLU). One hundred ninety-two samples collected as part of a Trichomonas vaginalis clinical study were used to challenge the C. trachomatis cryptic plasmid and intergenic spacer PCR assays described in Examples 3 and 4. Genomic DNA extracted from sample collection swab eluates were tested in both the cryptic plasmid and intergenic spacer assays as part of sensitivity and specificity testing for the assays. Concordance between the GenProbe Aptima Combo 2® Assay and both the cryptic plasmid and intergenic spacer assays was 100%. The intergenic spacer and cryptic plasmid assays accurately detected the 5/192 samples that were positive for C. trachomatis in the GenProbe assay. Two of those samples were also co-infected with T. vaginalis.

Mean cycle-threshold (Ct) values were highly correlated (Spearman's Rho=0.99) with a P value of less than 0.01 in a paired t-test. Samples from women who had clearly documented mucopurulent cervicitis were detected at a lower Ct value using the cryptic plasmid assay when compared to the intergenic spacer assay. These results are summarized in Table 4.

In addition, the intergenic spacer and cryptic plasmid assays described herein were further validated in a set of 400 clinical samples from commercial sex workers from Mombasa, Kenya. Test results were compared to GenProbe APTMA COMBO 2 assay test results and showed excellent concordance (data not shown).

TABLE 4
Threshold of detection among concordant positive clinical samples by
cryptic plasmid (CP) and intergenic spacer (IS) assays.

| | | Mean Ct | | |
|---|---|---|---|---|
| | | Values: | Mean Ct | GenProbe |
| Clinical | | Cryptic | Values: | Aptima test |
| Sample | Clinical | Plasmid | Intergenic | results |
| ID | Staging | Assay | Spacer Assay | (RLUs) |
| 51 | Asymptomatic | 30.2 | 34.2 | (826), positive |
| 181 | Asymptomatic | 30.1 | 34.1 | (956), positive |
| 19 | Cervicitis | 26.6 | 29.9 | (708), positive |
| 184 | Cervicitis | 28.3 | 31.6 | (1045), positive |
| 185 | Cervicitis | 22.4 | 25.8 | (924), positive |

The data shown in Table 4 illustrate the ability of the intergenic spacer and cryptic plasmid assays of the invention to robustly detect C. trachomatis infection in clinical samples, some of which are co-infected with T. vaginalis.

### Example 7

In this Example a number of samples known to be positive for the presence of C. trachomatis by culture and representing different serovars were tested in both the intergenic spacer assay, carried out as described in Example 3, and the cryptic plasmid assay, carried out as described in Example 4. The results illustrate the sensitivity of the assays in detecting the presence of C. trachomatis isolated from both cervical and urethral samples and represent a range of serovars commonly found in C. trachomatis infections in the US. The results are shown in Table 5.

TABLE 5
Sensitivity of cryptic plasmid assay and intergenic spacer assay

| | | | Detected | | Detected | |
|---|---|---|---|---|---|---|
| | | | in | Sensi- | in | Sensi- |
| | Site of | | cryptic | tivity* | intergenic | tivity* |
| | collec- | CT | plasmid | (EB/ | spacer | (EB/ |
| ID # | tion | serovar | assay | rxn) | assay | rxn) |
| PTH 002 | Urethra | K | + | <20 | + | <20 |
| PTH 003 | Cervix | F | + | <20 | + | <20 |
| PTH 004 | Cervix | 1a | + | <20 | + | <20 |
| PTH 005 | Cervix | E | + | <20 | + | <20 |
| PTH 006 | Urethra | E | + | <20 | + | <20 |
| PTH 008 | Urethra | 1a | + | <20 | + | <20 |
| PTH 009 | Cervix | K | + | <20 | + | <20 |
| PTH 010 | Urethra | F | + | <20 | + | <20 |
| PTH 011 | Urethra | G | + | <20 | + | <20 |
| PTH 012 | Urethra | H | + | <20 | + | <20 |
| PTH 013 | Urethra | J | + | <20 | + | <20 |
| PTH 014 | Cervix | J | + | <20 | + | <20 |
| PTH 015 | Urethra | D | + | <20 | + | <20 |
| PTH 016 | Cervix | D | + | <20 | + | <20 |
| PTH 017 | Cervix | H | + | <20 | + | <20 |
| PTH 018 | Cervix | G | + | <20 | + | <20 |

*The amounts shown above are an approximation and are expressed as EBs/reaction.

The values are based on counts of live EBs only. As a result, the numbers of dead EBs which also impact the amount of input template for the PCR reactions are not reflected.

### Example 8

In this Example a number of organisms which may be found at the site of swab collection, and organisms closely related to C trachomatis, were tested in both the intergenic spacer assay, carried out as described in Example 3, and the cryptic plasmid assay, carried out as described in Example 4, to illustrate the specificity of the assays for C. trachomatis. The results are shown in Table 6.

TABLE 6
Summary of specificity testing with near neighbor organisms and
organisms found in same biological site.

| | Detected in | | | |
|---|---|---|---|---|
| | cryptic | Sensitivity | Detected in | Sensitivity |
| | plasmid | (amount | intergenic | (amount |
| Organism | assay | DNA/rxn) | spacer assay | DNA/rxn) |
| Streptococcus | Negative | | Negative | |
| group B | | | | |
| Streptococcus | Negative | | Negative | |
| pyrogenes | | | | |
| E. coli | Negative | | Negative | |
| Staphlococcus | Negative | | Negative | |
| aureus | | | | |
| Entamoeba faecalis | Negative | | Negative | |
| S. epididymis | Negative | | Negative | |
| Murine | Negative | 50 ng* | Negative | 50 ng* |
| pneumocystis | | | | |
| (MoPn) | | | | |
| C. psittaci | Negative | 50 ng* | Negative | 50 ng* |
| C. pneumoniae | Positive | 50 ng | Positive | 50 ng |

*Results were negative when tested with 50 ng template per reaction but were positive when tested with amounts of 100 ng DNA or greater per reaction. This is reflective of the degree of sequence homology between the target genes of the organisms.

The test results illustrate that the cryptic plasmid assay and intergenic spacer assay of the invention are specific for detecting C. trachomatis as opposed to other organisms that could be potential contaminants during sample collection. The closely related species of C. psittaci and C. pneumoniae are detected by both the cryptic plasmid assay and intergenic spacer assay but only at levels of input template that greatly exceed that which could be found in a clinical sample.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for determining the presence of Chlamydia trachomatis in a test sample, said method comprising the steps of:
(a) contacting a test sample with a composition comprising at least one primer pair comprising a forward primer and a reverse primer capable of hybridizing to a target region of the C. trachomatis intergenic spacer sequence consisting of SEQ ID NO:2 to form a reaction mixture; and
(b) subjecting said reaction mixture to amplification conditions suitable to amplify at least a portion of said target region.

2. The method of claim 1, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

3. The method of claim 1, wherein the composition comprises a primer having a target-binding region consisting of SEQ ID NO:3.

4. The method of claim 1, wherein the composition comprises a primer having a target-binding region consisting of SEQ ID NO:4.

5. The method of claim 2, wherein the detection probe comprises a target-binding region consisting of SEQ ID NO:5.

6. method of claim 2, wherein determining the presence of said hybrid in said reaction mixture indicates the presence of C. trachomatis in said test sample.

7. A method for determining the presence of C. trachomatis in a test sample, said method comprising the steps of:
(a) contacting a test sample with a composition comprising at least one primer pair comprising a forward primer and a reverse primer capable of hybridizing to a target region of the C. trachomatis cryptic plasmid consisting of SEQ ID NO:7 to form a reaction mixture; and
(b) subjecting said reaction mixture to amplification conditions suitable to amplify at least a portion of said target region.

8. The method of claim 7, further comprising detecting the presence of the amplified portion by contacting the reaction mixture with a detection probe under hybridizing conditions, wherein the detection probe has a nucleotide sequence that hybridizes to at least a portion of the amplified target region and determining the presence of a hybrid.

9. The method of claim 7, wherein the composition comprises a primer having a target-binding region consisting of SEQ ID NO:8.

10. The method of claim 7, wherein the composition comprises a primer having a target-binding region consisting of SEQ ID NO:9.

11. The method of claim 8, wherein the detection probe comprises a target-binding region consisting of SEQ ID NO:10.

12. The method of claim 8, wherein determining the presence of said hybrid in said reaction mixture indicates the presence of C. trachomatis in said test sample.

13. A method for determining the presence of C. trachomatis in a test sample, said method comprising the steps of:
(a) contacting a test sample with a composition comprising at least one primer pair comprising a forward primer and a reverse primer capable of hybridizing to a target region of the C. trachomatis intergenic spacer sequence consisting of SEQ ID NO:2 and at least one primer pair comprising a forward primer and a reverse primer capable of hybridizing to a target region of the C. trachomatis cryptic plasmid consisting of SEQ ID NO:7 to form a reaction mixture; and
(b) subjecting said reaction mixture to amplification conditions suitable to amplify at least a portion of said target region.

14. A set of oligonucleotides for use in amplifying a target region of nucleic acid derived from the intergenic spacer sequence of C. trachomatis, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:2.

15. The set of oligonucleotides of claim 14, wherein the forward primer consists of SEQ. ID NO:3.

16. The set of oligonucleotides of claim 14, wherein the reverse primer consists of SEQ ID NO:4.

17. The set of oligonucleotides of claim 14, further comprising a detection probe consisting of SEQ ID NO:5.

18. A set of oligonucleotides for use in amplifying a target region of nucleic acid derived from the C. trachomatis cryptic plasmid, the set of oligonucleotides comprising a forward primer and a reverse primer, each primer having a target binding region up to 30 nucleotides in length, which contains at least 10 contiguous nucleotides that are perfectly complementary to an at least 10 contiguous nucleotide region present in a target sequence consisting of SEQ ID NO:7.

19. The set of oligonucleotides of claim 18, wherein the forward primer consists of SEQ ID NO:8.

20. The set of oligonucleotides of claim 18, wherein the reverse primer consists of SEQ ID NO:9.

21. The set of oligonucleotides of claim 18, further comprising a detection probe consisting of SEQ ID NO:10.

22. An oligonucleotide for use in amplifying a target region of nucleic acid derived from C. trachomatis, said oligonucleotide having a target binding region of up to 30 bases in length, which stably hybridizes to a target sequence selected from the group consisting of SEQ ID NO:2 and SEQ ID NO:7.

23. The oligonucleotide of claim 22, wherein said target binding region contains at least 10 contiguous nucleotides that are perfectly complementary to at least 10 contiguous nucleotides in said target sequence.

24. The oligonucleotide of claim 22, wherein said target region consists of SEQ ID NO:2.

25. The oligonucleotide of claim 24, wherein said target binding region consists of SEQ ID NO:3, SEQ ID NO:4, or SEQ ID NO:5.

26. The oligonucleotide of claim 22, wherein said target region consists of SEQ ID NO:7.

27. The oligonucleotide of claim 26, wherein said target binding region consists of SEQ ID NO:8, SEQ ID NO:9, or SEQ ID NO:10.

28. A kit for detecting the presence of C. trachomatis in a test sample, the kit comprising:
(a) at least one oligonucleotide comprising a target binding region sequence selected from the group consisting of SEQ ID NO:3, SEQ ID NO:4, SEQ ID NO:5, SEQ ID NO:8, SEQ ID NO:9, and SEQ ID NO:10;
(b) amplification reagents; and
(c) written instructions describing amplification conditions suitable to detect the presence of C. trachomatis in a test sample in the test sample.

29. The kit of claim 28, wherein one or more of the oligonucleotides incorporates one or more detectable labels.


SEQUENCE LISTING (10 sequences)

SEQ ID NO: 1 - DNA, 1304 bp, Chlamydia trachomatis
catgaagtcggaattgctagtaatggcgtgtcagccataacgccgtgaatacgttcccgggccttgtacacaccgcccgtcacatcatgggagttggttttaccttaagtcgttgactcaacccgcaagggagagaggcgcccaaggtgaggctgatgactaggatgaagtcgtaacaaggtagccctaccggaaggtggggctggatcacctccttttaaggataaggaagaagcctgagaaggtttctgactaggttgggcaagcgtttatatgtaagagcaagcattctatttcatttgtgttgttaagagtagcgcggtgaggacgagacatatagtttgtgatcaagtatgttattgtaaagaaataatcatggtaacaagtatatttcacgcataataatagacgtttaagagtatttgtcttttaggtgaagtgcttgcatggatctatagaaattacagaccaagttaataagagctattggtggatgccttggcattgacaggcgaagaaggacgcgaatacctgcgaaaagctccggcgagctggtgataagcaaagacccggaggtatccgaatggggaaacccggtagagtaatagactaccattgcatgctgaatacataggtatgcaaagcgacacctgccgaactgaaacatcttagtaagcagaggaaaagaaatcgaagagattccctgtgtagcggcgagcgaaaggggaatagcctaaaccgagctgataaggctcggggttgtaggattgaggataaaggatcaggactcctagttgaacacatctggaaagatggatgatacagggtgatagtcccgtagacgaaaggagagaaagaccgacctcaacacctgagtaggactagacacgtgaaacctagtctgaatctggggagaccactctccaaggctaaatactagtcaatgaccgatagtgaaccagtactgtgaaggaaaggcgaaaagaacccttgttaagggagtgaaatagaacctgaaaccagtagcttacaagcggtcggagaccaatggcccgtaagggtcaaggttgacggcgtgccttttgcatgatgagccagggagttaagctaaacggcgaggttaagggatatacattccggagccggagcgaaagcgagttttaaaagagcgaagagtcgtttggtttagacacgaaaccaagtgagctatttatgaccaggttgaagcatgggtaaaactatgtggaggaccgaactagtacctgttgaaaaaggtttggatgagttgtgaataggggtgaaag

SEQ ID NO: 2 - DNA, 290 bp, Chlamydia trachomatis
gagttggttttaccttaagtcgttgactcaacccgcaagggagagaggcgcccaaggtgaggctgatgactaggatgaagtcgtaacaaggtagccctaccggaaggtggggctggatcacctccttttaaggataaggaagaagcctgagaaggtttctgactaggttgggcaagcgtttatatgtaagagcaagcattctatttcatttgtgttgttaagagtagcgcggtgaggacgagacatatagtttgtgatcaagtatgttattgtaaagaaataatcatggt

SEQ ID NO: 3 - DNA, 31 bp, Artificial sequence
  Synthetic
gagttggttttaccttaagtcgttgactcaa

SEQ ID NO: 4 - DNA, 36 bp, Artificial sequence
  Synthetic
accatgattatttctttacaataacatacttgatca

SEQ ID NO: 5 - DNA, 26 bp, Artificial sequence
  Synthetic
caaggtgaggctgatgactaggatga

SEQ ID NO: 6 - DNA, 7500 bp, Chlamydia trachomatis
tttgcaactcttggtggtagactttgcaactcttggtggtagactttgcaactcttggtggtagactttgcaactcttggtggtagacttggtcataatggacttttgttgaaaaatttcttaaaatcttagagctccgattttgaatagctttggttaagaaaatgggctcgatggctttccataaaagtaggttgttcttaacttttggggacgcgtcggaaatttggttatctactttatctcatctaactagaaaaaattatgcgtctgggattaactttcttgtttctttagagattctggatttatcggaaaccttgataaaggctatttctcttgaccacagcgaatctttgtttaaaatcaagtctctagatgtttttaatggaaaagtcgtttcagaggcctctaaacaggctagagcggcatgctacatatctttcacaaagtttttgtatagattgaccaagggatatattaaacccgctattccattgaaagattttggaaacactacattttttaaaatccgagacaaaatcaaaacagaatcgatttctaagcaggaatggacagttttttttgaagcgctccggatagtgaattatagagactatttaatcggtaaattgattgtacaagggatccgtaagttagacgaaattttgtctttgcgcacagacgatctattttttgcatccaatcagatttcctttcgcattaaaaaaagacagaataaagaaaccaaaattctaatcacatttcctatcagcttaatggaggagttgcaaaaatacacttgtgggagaaatgggagagtatttgtttctaaaatagggattcctgtaacaacaagtcaggttgcgcataattttaggcttgcagagttctatagtgctatgaaaataaaaattactcctagagtacttcgtgcaagcgctttgattcatttaaagcaaataggattaaaagatgaggaaatcatgcgtatttcctgtctttcatcgagacaaagtgtgtgttcttattgttctggggaagaggtaagtcctctagtacaaacacccccaatattgtgatataattaaaattatattcatattctgttgccagaaaaaacacttttaggctatattagagccatcttctttgaagcgttgtcttctcgagaagatttatcgtacgcaaatatcatctttgcggttgcgtgtcctgtgaccttcattatgtcggagtctgagcaccctaggcgtttgtactccgtcacagcggttgctcgaagcacgtgcggggttatcttaaaagggattgcagcttgtagtcctgcttgagagaacgtgcgggcgatttgccttaaccccaccatttttccggagcgagttacgaagacaaaacctcttcgttgaccgatgtactcttgtagaaagtgcataaacttctgaggataagttataataatcctcttttctgtctgacggttcttaagctgggagaaagaaatggtagcttgttggaaacaaatctgactaatctccaagcttaagacttcagaggagcgtttacctccttggagcattgtctgggcgatcaaccaatcccgggcattgattttttttagctcttttaggaaggacgctgtttgcaaactgttcatcgcatctgtttttactatttccctggttttaaaaaatgttcgactattttcttgtttagaaggttgcgctatagcgactattccttgagtcatcctgtttaggaatcttgttaaggaaatatagcttgctgctcgaacttgtttagtaccttcggtccaagaagtcttggcagaggaaacttttttaatcgcatctagaattagattatgatttaaaagggaaaactcttgcagattcatatccaaggacaatagaccaatcttttctaaagacaaaaaagatcctcgatatgatctacaagtatgtttgttgagtgatgcggtccaatgcataataacttcgaataaggagaagcttttcatgcgtttccaataggattcttggcgaatttttaaaacttcctgataagacttttcgctatattctaacgacatttcttgctgcaaagataaaatccctttacccatgaaatccctcgtgatataacctatccgtaaaatgtcctgattagtgaaataatcaggttgttaacaggatagcacgctcggtatttttttatataaacatgaaaactcgttccgaaatagaaaatcgcatgcaagatatcgagtatgcgttgttaggtaaagctctgatatttgaagactctactgagtatattctgaggcagcttgctaattatgagtttaagtgttctcatcataaaaacatattcatagtatttaaatacttaaaagacaatggattacctataactgtagactcggcttgggaagagcttttgcggcgtcgtatcaaagatatggacaaatcgtatctcgggttaatgttgcatgatgctttatcaaatgacaagcttagatccgtttctcatacggttttcctcgatgatttgagcgtgtgtagcgctgaagaaaatttgagtaatttcattttccgctcgtttaatgagtacaatgaaaatccattgcgtagatctccgtttctattgcttgagcgtataaagggaaggcttgacagtgctatagcaaagactttttctattcgcagcgctagaggccggtctatttatgatatattctcacagtcagaaattggagtgctggctcgtataaaaaaaagacgagcaacgttctctgagaatcaaaattctttctttgatgccttcccaacaggatacaaggatattgatgataaaggagttatcttagctaaaggtaatttcgtgattatagcagctaggccatctatagggaaaactgctttagctatagacatggcgataaatcttgcggttactcaacagcgtagagttggtttcctatctctagaaatgagcgcaggtcaaattgttgagcggattattgctaatttaacaggaatatctggtgaaaaattacaaagaggggatctctctaaagaagaattattccgagtagaagaagctggagaaacagttagagaatcacatttttatatctgcagtgatagtcagtataagcttaatttaatcgcgaatcagatccggttgctgagaaaagaagatcgagtagacgtaatatttatcgattacttgcagttgatcaactcatcggttggagaaaatcgtcaaaatgaaatagcagatatatctagaaccttaagaggtttagcctcagagctaaacattcctatagtttgcttatcccaactatctagaaaagttgaggatagagcaaataaagttcccatgctttcagatttgcgagacagcggtcaaatagagcaagacgcagatgtgattttgtttatcaataggaaggaatcgtcttctaattgtgagataactgttgggaaaaatagacatggatcggttttctcttcggtattacatttcgatccaaaaattagtaaattctccgctattaaaaaagtatggtaaattatagtaactgccacttcatcaaaagtcctatccaccttgaaaatcagaagtttggaagaagacctggtcaatctattaagatatctcccaaattggctcaaaatgggatggtagaagttataggtcttgattttctttcatctcattaccatgcattagcagctatccaaagattgctgactgcaacgaattacaaggggaacacaaaaggggttgttttatccagagaatcaaatagttttcaatttgaaggatggataccaagaatccgttttacaaaaactgaattcttagaggcttatggagttaagcggtataaaacatccagaaataagtatgagtttagtggaaaagaagctgaaactgctttagaagccttataccatttaggacatcaaccgtttttaatagtggcaactagaactcgatggactaatggaacacaaatagtagaccgttaccaaactctttctccgatcattaggatttacgaaggatgggaaggtttaactgacgaagaaaatatagatatagacttaacaccttttaattcaccatctacacggaaacataaagggttcgttgtagagccatgtcctatcttggtagatcaaatagaatcctactttgtaatcaagcctgcaaatgtataccaagaaataaaaatgcgcttcccaaatgcatcaaagtatgcttacacatttatcgactgggtgattacagcagctgcgaaaaagagacgaaaattaactaaggataattcttggccagaaaacttgttcttaaacgttaacgttaaaagtcttgcatatattttaaggatgaatcggtacatttgtacaaggaactggaaaaaaatcgagttagctatcgataaatgtatagaaatcgccattcagcttggttggttatctagaagaaaacgcattgaatttctggattcttctaaactctctaaaaaagaaattctatatctaaataaagagcgttttgaagaaataaccaagaaatctaaagaacaaatggaacaattagaacaagaatctattaattaatagcaaacttgaaactaaaaacctaatttatttaaagctcaaaataaaaaagagttttaaaatgggaaattctggtttttatttgtataacactcaaaactgcgtctttgctgataatatcaaagttgggcaaatgacagagccgctcaaggaccagcaaataatccttgggacaacatcaacacctgtcgcagccaaaatgacagcttctgatggaatatctttaacagtctccaataatccatcaaccaatgcttctattacaattggtttggatgcggaaaaagcttaccagcttattctagaaaagttgggagatcaaattcttggtggaattgctgatactattgttgatagtacagtccaagatattttagacaaaatcacaacagacccttctctaggtttgttgaaagcttttaacaactttccaatcactaataaaattcaatgcaacgggttattcactcccaggaacattgaaactttattaggaggaactgaaataggaaaattcacagtcacacccaaaagctctgggagcatgttcttagtctcagcagatattattgcatcaagaatggaaggcggcgttgttctagctttggtacgagaaggtgattctaagccctacgcgattagttatggatactcatcaggcgttcctaatttatgtagtctaagaaccagaattattaatacaggattgactccgacaacgtattcattacgtgtaggcggtttagaaagcggtgtggtatgggttaatgccctttctaatggcaatgatattttaggaataacaaatacttctaatgtatcttttttggaggtaatacctcaaacaaacgcttaaacaatttttattggatttttcttataggttttatatttagagaaaaaagttcgaattacggggtttgttatgcaaaataaaagcaaagtgagggacgattttattaaaattgttaaagatgtgaaaaaagatttccccgaattagacctaaaaatacgagtaaacaaggaaaaagtaactttcttaaattctcccttagaactctaccataaaagtgtctcactaattctaggactgcttcaacaaatagaaaactctttaggattattcccagactctcctgttcttgaaaaattagaggataacagtttaaagctaaaaaaggctttgattatgcttatcttgtctagaaaagacatgttttccaaggctgaatagataacttactctaacgttggagttgatttgcacaccttagttttttgctcttttaagggaggaactggaaaaacaacactttctctaaacgtgggatgcaacttggcccaatttttagggaaaaaagtgttacttgctgacctagacccgcaatccaatttatcttctggattgggggctagtgtcagaagtaaccaaaaaggcttacacgacatagtatacacatcaaacgatttaaaatcaatcatttgcgaaacaaaaaaagatagtgtggacctaattcctgcatcatttttatccgaacagtttagagaattggatattcatagaggacctagtaacaacttaaagttatttctgaatgagtactgcgctcctttttatgacatctgcataatagacactccacctagcctaggagggttaacgaaagaagcttttgttgcaggagacaaattaattgcttgtttaactccagaacctttttctattctagggttacaaaagatacgtgaattcttaagttcggtcggaaaacctgaagaagaacacattcttggaatagctttgtctttttgggatgatcgtaactcgactaaccaaatgtatatagacattatcgagtctatttacaaaaacaagcttttttcaacaaaaattcgtcgagatatttctctcagccgttctcttcttaaagaagattctgtagctaatgtctatccaaattctagggccgcagaagatattctgaagttaacgcatgaaatagcaaatattttgcatatcgaatatgaacgagattactctcagaggacaacgtgaacaaactaaaaaaagaagcgaatgtcttttttaaaaaaaatcaaactgccgcttctttagattttaagaagacgcttccttccattgaactattctcagcaactttgaattctgaggaaagtcagagtttggatcaattatttttatcagagtcccaaaactattcggatgaagaattttatcaagaagacatcctagcggtaaaactgcttactggtcagataaaatccatacagaagcaacacgtacttcttttaggagaaaaaatctataatgctagaaaaatcctgagtaaggatcacttctcctcaacaactttttcatcttggatagagttagtttttagaactaagtcttctgcttacaatgctcttgcatattacgagctttttataaacctccccaaccaaactctacaaaaagagtttcaatcgatcccctataaatccgcatatattttggccgctagaaaaggcgatttaaaaaccaaggtcgatgtgatagggaaagtatgtggaatgtcgaactcatcggcgataagggtgttggatcaatttcttccttcatctagaaacaaagacgttagagaaacgatagataagtctgattcagagaagaatcgccaattatctgatttcttaatagagatacttcgcatcatgtgttccggagtttctttgtcctcctataacgaaaatcttctacaacagctttttgaactttttaagcaaaagagctgatcctccgtcagctcatatatatatctattatatatatatatttagggatttgattttacgagagaga

SEQ ID NO: 7 - DNA, 69 bp, Chlamydia trachomatis
ttggctcaaaatgggatggtagaagttataggtcttgattttctttcatctcattaccatgcattagca

SEQ ID NO: 8 - DNA, 20 bp, Artificial sequence
  Synthetic
ttggctcaaaatgggatggt

SEQ ID NO: 9 - DNA, 24 bp, Artificial sequence
  Synthetic
tgctaatgcatggtaatgagatga

SEQ ID NO: 10 - DNA, 20 bp, Artificial sequence
  Synthetic
agttataggtcttgattttc